(19) United States
(12) Patent Application Publication
(10) Pub. No.: US 2005/0132426 A1
(43) Pub. Date: Jun. 16, 2005

(54) Long-term cell culture compositions and genetically modified animals derived therefrom

(76) Inventors: John Roderick Morrison, Carnegie (AU); Eric Shannon Hayes, Victoria (CA); Martin Frederick Pera, Prahran (AU); Orly Lacham-Kaplan, East Bentleigh (AU); Alan Osborne Trounson, Ashburton (AU)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/041,152

(22) Filed: Jan. 21, 2005

Related U.S. Application Data
(63) Continuation of application No. 09/732,520, filed on Dec. 7, 2000, now abandoned.

(30) Foreign Application Priority Data
Dec. 7, 1999 (AU) PQ4495
Aug. 7, 2000 (AU) PQ9242
Oct. 31, 2000 (AU) PR1108
Oct. 31, 2000 (AU) PR1109

(51) Int. Cl. A01K 67/00; C12N 5/06; C12N 5/08

(52) U.S. Cl. 800/8; 435/368; 435/354

(57) ABSTRACT

The present invention generally relates to neural stem cells, preferably fetal neural stem cells and their progeny thereof. The present invention provides methods of isolating culturing and propagating neural stem cells and the development of neural stem cell lines and lineages. The present invention also relates to the use of neural stem cells and somatic cells and cells expressing the telomerase catalytic component (TERT) for gene targeting and gene knockout experiments and for producing genetically modified animals.

The present invention generally relates to neural stem cells, preferably foetal neural stem cells and their progeny thereof. The present invention provides methods of isolating, culturing and propagating neural stem cells preferably foetal neural stem cells and the development of neural stem cell lines and lineages. The present invention also relates to the use of neural stem cells and somatic cells (eg rat fetal fibroblasts) and cells expressing the telomerase catalytic component (TERT) for gene targeting and gene knockout experiments and for producing genetically modified animals.

### INTRODUCTION

The characterisation and isolation of neural stem cells is useful to understand and treat neurological disorders in mammals. In addition, cell lines based on neural stem cells may be suitable for gene targeting and gene knockout experiments and for nuclear transfer experiments to produce genetically modified animals.

Foetal neural stem (FNS) cells area heterogenous population of glial, astrocyte and neuronal progenitor cells that are capable of differentiating into a variety cell types including neurons. A neural stem cell is an undifferentiated cell that is capable of differentiating into one or more different types of cells. Such stem cells are characterised by having the ability to proliferate, differentiate and are capable of self-renewal. These cells may be derived from various tissues, including the brain and/or spinal cord of the embryonic or adult central nervous system.

However, it has been difficult to obtain a neural stem cell line that has the capacity to remain robust and allow for self-renewal and further differentiate in vitro.

Several attempts to isolate neural stem cells have been made. U.S. Pat. No. 5,928,947 reports methods of isolating and clonal propagation of neural crest stem cells isolated, from embryonic tissue. U.S. Pat. No. 6,040,180 reports the short-term propagation (20 days) of rat embryonic stem cells. The source of these specific types of neural stem cells and the methods taught to culture the particular cells are applicable to embryonic tissue. However none of these patents describe or claim, the ability to be able to maintain long-term cultures of rat foetal neural stems cells.

Therefore, although, culture systems and cell lines have been established from neural stem cells isolated from embryos, it is desirable to develop a neural stem, cell line derived from foetal tissue with long-term growth potential. The neural stem population isolated at this later stage of development has a different phenotype and characteristics to embryonic stem cells. Neural stem cells isolated from foetal tissue are easy to isolate and grow.

The advantage of using neural stem cells is that they are believed to have a greater degree of developmental plasticity and therefore have the ability to generate neural lineages and haematopoietic lineages etc. Therefore, due to the multipotent phenotype of neural stem cells and their ability to readily multiply in a suitable culture they are useful for gene targeting and gene knockout experiments. It would be desirable to develop neural stem cells for gene targeting and gene knockout experiments. Developmental abnormalities associated with nuclear transfer technology using somatic cells have been reported. This results in a high rate of mortality either in utero or perinatally. While it is unclear what is causing these defects it is possible that the further a cell has progressed along a differentiation pathway (ie the cells are less plastic) the less able the cell is capable of being reprogrammed. This must occur for cloning technologies to be successful.

The successful development of normal animals from a number of mammalian species using somatic cell nuclear transfer techniques has lead to the possibility that this approach may be used for the production of large numbers of genetically modified livestock and animals for biomedical research. However, one of the major limitations to this technology is found in the normal life span of the somatic cells generally used as the source of donor nuclei in the nuclear transfer procedures. Mammalian somatic cells have a limited life span and enter senescence after a limited number of cell divisions. Because the successful integration or deletion of a DNA sequence in cells in culture requires a relatively large number of cellular divisions, this limit on cell proliferation represents an obstacle to the genetic manipulation of the donor cell nuclei and, ultimately, to the production of genetically modified animals by nuclear transfer. The production of somatic cells capable of continuous growth in culture and their application to nuclear transfer would represent a major step towards the production of such genetically modified animals. One method for overcoming the limitations of senescence is to stably incorporate the catalytic component of telomerase (TERT) into a cell. Methods for the incorporation of TERT and the consequent characteristics of such cells have previously been reported in U.S. Pat. No. 5,981,707 and U.S. Pat. No. 5,958,680.

The discussion of documents, acts, materials, devices, articles and the like is included in this description solely for the purpose of providing a context for the at present invention. It is not suggested or represented that any or all of these matters formed, part of the prior art base or were common general knowledge in the field relevant to the present invention as it existed in Australia.

Accordingly, it is an object of the present invention to overcome or at least alleviate some of the problems with the prior art and to provide a cellular composition which supports culturing of neural stem cells for long-term culture and to develop cells capable of long-term culture.

### SUMMARY OF THE INVENTION

In a first aspect of the present invention there is provided a cellular composition comprising one or more cells having a property characteristic of a neural stem cell and wherein said neural stem cell is capable of long term culture. Preferably the cells have a property characteristic of a foetal neural stem cell.

In another aspect of the present invention, there is provided a method of preparing a cellular composition comprising a substantially homogeneous population of cells having a property characteristic of a neural stem cell and wherein said neural stem cell is capable of long term culture said method comprising:
obtaining a source of neural stem cells;
preparing a suspension of cells from the source;
contacting the suspension of cells with a suitable medium to maintain the neural stem cells in a cell culture;
culturing the cells including passaging and propagation of cells.

In another aspect of the present invention, there is provided a media suitable for culturing NSC's, said media including at least one lipid and at least one mitogenic factor in said media.

In yet another aspect there is provided a method of culturing neural stem cells in long term culture, said method comprising culturing the cells in the presence of at least one lipid and at least one mitogenic factor.

In another aspect of the present invention, there is provided a genetically modified neural stem cell capable of long term culture, said cell comprising a foreign gene which has been introduced into the neural stem cell.

In another aspect of the present invention, there is provided a genetically modified neural stem cell capable of long term culture, said cell having a destroyed, modified or deleted gene. Such genetically modified neural stem cells are useful in gene targeting and gene knockout experiments.

In another aspect of the present invention there is provided a method of producing an animal, said method comprising introducing a continuously growing donor cell nucleus from a continuously growing donor cell into an oocyte or embryo and allowing the resulting embryo to mature and to preferably develop to a foetus or an adult animal.

In a preferred aspect of the present invention, the donor cell is a genetically modified somatic cell. Preferably, the donor cell is derived from a non-transformed immortalised cell line that expresses telomerase catalytic component (TERT), which allows the cell to grow continuously in culture thereby enabling repeated genetic manipulations of the cell. Similarly, the nucleus may be derived from the immortalized cell line or genetically modified somatic cell which is continuously growing.

In another preferred aspect of the present invention, the donor cell is a further genetically modified TERT cell, said TERT cell comprising a foreign gene which has been introduced into a somatic cell.

In another preferred aspect, the nucleus is derived from a genetically modified TERT cell comprising a foreign gene which has been introduced into the a somatic.

In yet another preferred aspect of the present invention, the donor cell is a further genetically modified TERT cell, said TERT cell having a destroyed, modified or deleted gene. Such genetically modified TERT cells are useful in gene targeting and gene knockout experiments.

In yet another preferred aspect, the nucleus is derived-from a further genetically modified TERT cell, said TERT cell having a destroyed, modified or deleted gene.

In another aspect of the present invention there is provided a method of producing a cell line that may be expanded from an embryo to produce cloned cells of an embryo, said method comprising
introducing a continuously growing donor cell or nucleus from a continuously growing cell, into an oocyte or embryo;
culturing the oocyte or embryo to an advanced cleavage stage embryo;
separating and cloning the cleaved cells of the embryo; and
optionally culturing the cloned cells.

In another aspect of the present invention there is provided an animal produced by the methods of the present invention. Preferably, the animal is a genetically modified animal, preferably the genetically modified animal is a knockout animal.

Preferably there is provided a method of preparing a genetically modified animal, said method comprising introducing a neural stem cell into an oocyte or embryo and allowing the resulting embryo to mature to a foetus or animal.

In another aspect of the invention, there is provided a method of treating a neurological disorder, said method comprising introducing a neural stem cell into a host animal to correct the disorder wherein the neural stem cell is capable of replacing neural cells affected by the neurological disorder.

The present invention further includes foetal neural stem cells isolated by the methods hereinbefore described which are transfected with exogenous nucleic acid or are genetically modified by destroying, modifying or deleting genes. Selected foreign nucleic acid may be introduced and/or recombinantly expressed in the cells of the present invention through the use of conventional techniques or the genes may be modified, destroyed or deleted by methods such as point or random mutations.

### FIGURES

FIG. 1 shows the neural stem cells form a multilayered culture displaying a number of morphologies depending on whether the cells are in direct contact with the tissue culture plate or are part of a secondary layer (FIG. 1A). Continued proliferation of the cells results in the formation of budding structures (FIG. 1B), which will eventually “hatch” generating balls of cells floating in the media. These balls can be cultured in suspension or disaggregated to for growing on tissue culture plates.

FIG. 2 shows that the cells are positive for a number of markers consistent with neural stem cells including nestin (FIG. 2A) and vimentin (FIG. 2B).

FIG. 3 shows A) B) phase contract images of FNS cells that have been allowed to differentiate by passaging at low density. The cells are positive for markers of differentiated neuronal stem cells. C) shows differentiated neuronal stem cells expressing G-FAP, which is a marker of glial cells, using immunofluorescence. D) shows differentiated cells expressing β-tubulin a marker consistent with neurones using immunofluorescence.

FIG. 4 shows the effect of bFGF (FGF2) on FNS cell proliferation. bFGF ranging in concentration from 0-50 ng/ml was applied to various passage FNS cells (ie passage 2-12). At early passage number the cells show some independence of added growth factors which is lost past passage #5. Optimal bFGF stimulated proliferation of FNS cells occurs at approximately 5 ng/ml.

FIG. 5 shows the effect of EGF on FNS cell proliferation. EGF ranging in concentration from 0-50 ng/ml was applied to various passage FNS cells (ie passage 2-12). At early passage number the cells show some independence of added growth factors which is lost past passage #5. Optimal bFGF stimulated proliferation of FNS cells occurs at approximately 5 ng/ml.

FIG. 6 shows the combined effect of EGF and bFGF on FNS cell proliferation: A) Low concentration and B) high concentration. The combined effect of EGF and bFGF was tested on FNS cells. An optimal concentration of 2-5 ng/ml was observed for each growth factor when used in combination.

FIG. 7 shows long-term culture of FNS cells in the presence of and absence of EGF or bFGF. While there appears to be some variation between the various passages it was generally noted that there was little added benefit to adding both EGF and bFGF over adding bFGF alone to the culture system. However the FNS cells appear to be more responsive to EGF in the early passages.

FIG. 8 shows the effect of lipid on the propagation of foetal neural stem cells. All cells were propagated in the standard Neurobasal A media (with supplements) in the presence or absence of the Chemically defined lipid concentrate (diluted 1:100).

FIG. 9 shows the characteristics of cells grown in either DMEM/F12 media or Neurobasal A (plus supplements) media with or without the addition of the chemically defined lipid supplement. A) DMEM/F12−lipid (10× magnification); B) DMEM/F12−lipid (32× magnification); C) DMEM/F12+lipid (10× magnification); D) DMEM/F12+lipid (20× magnification); E) Neurobasal A−lipid (10× magnification) F) Neurobasal A−lipid (32× magnification); G) Neurobasal A+lipid (10× magnification); H) Neurobasal A+lipid (20× magnification)

FIG. 10 shows assessment of FNS cell proliferation using BrdU incorporation at 160× magnification. A) and —C) shows BrdU incorporation into passage #2: and passage #17 cells, respectively; BrdU incorporation is visualised using an mouse monoclonal anti-BrdU (Sigma) in combination with FITC conjugated goat anti-mouse. Photos are paired-there is one shot of BrdU immunofluorescence A) and C), and one shot of the same-cells using phase contrast microscopy B)- and D).

FIG. 11 shows the histology of tumours formed by the injection of PC12 cells (a neuronal cell tumour line) into SCID mice. Tissues were collected 19 days after injection and stained with H&E. The tumour morphology is consistent with neuroblastoma SCID mice injected with FNS cells (passage # 12) failed to display any signs of tumour formation after 13 weeks.

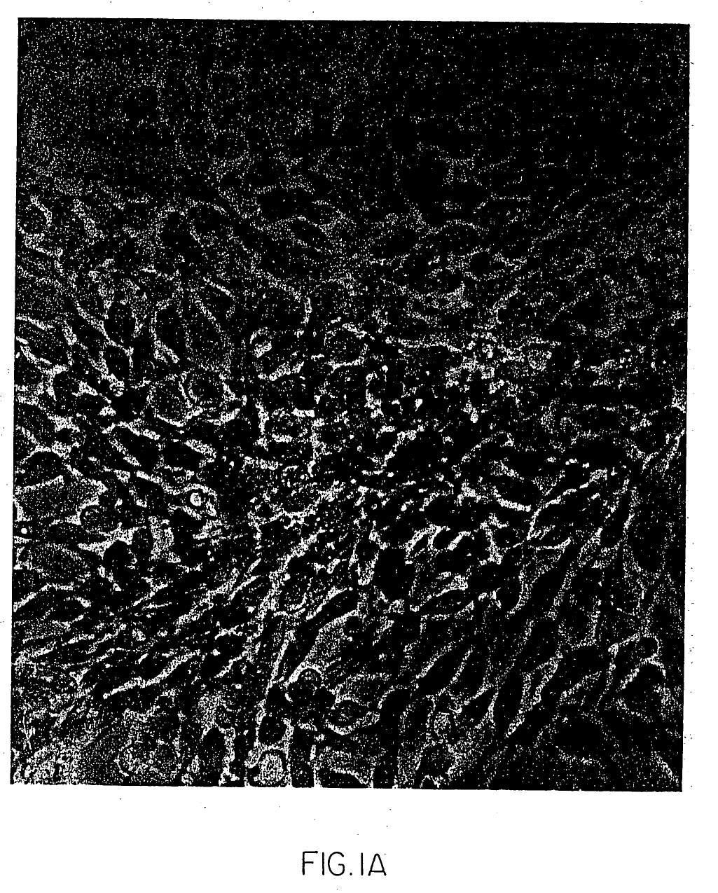

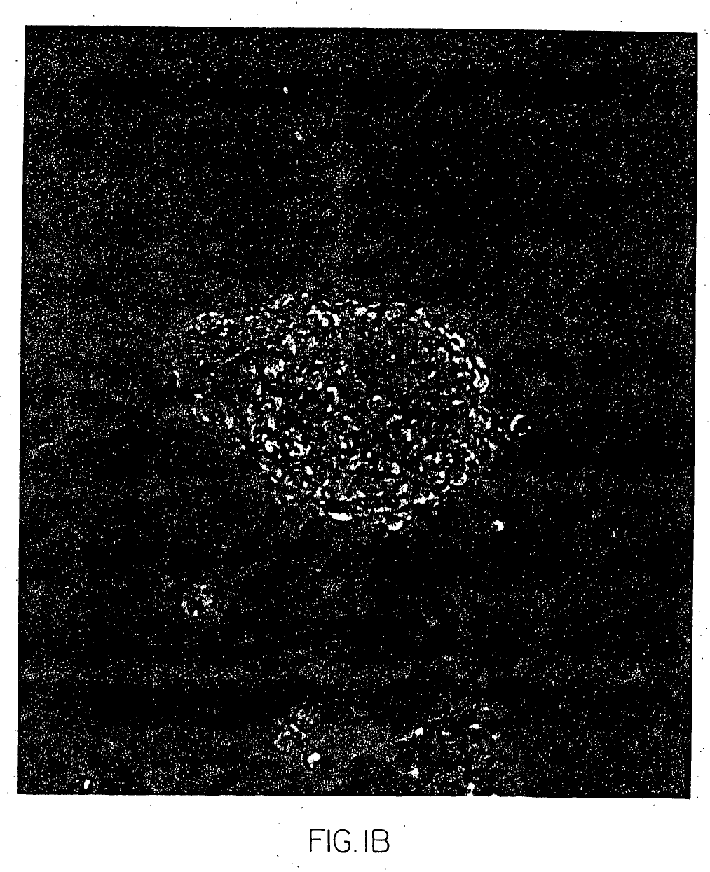

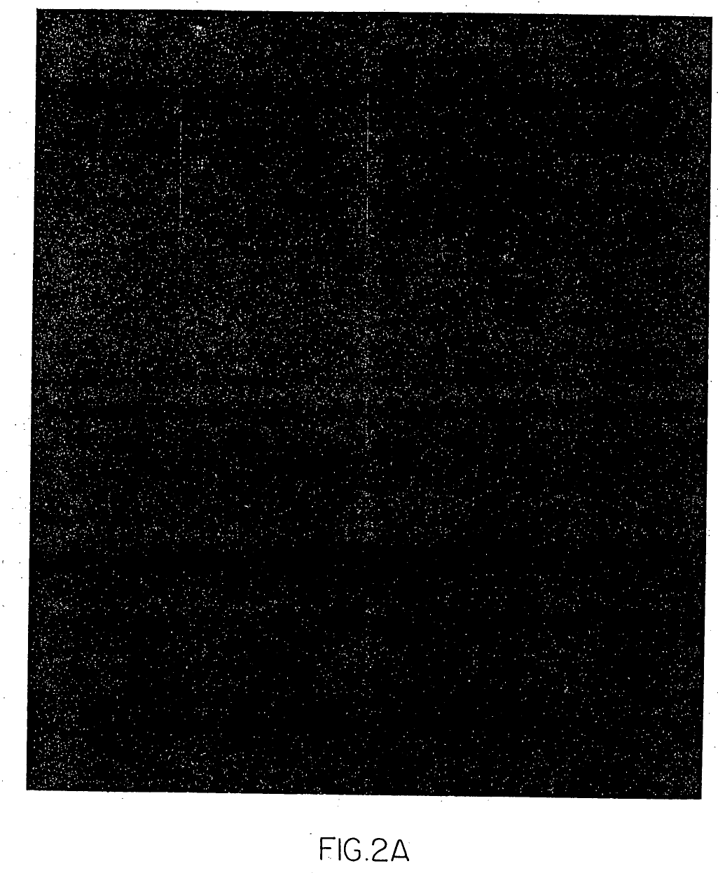

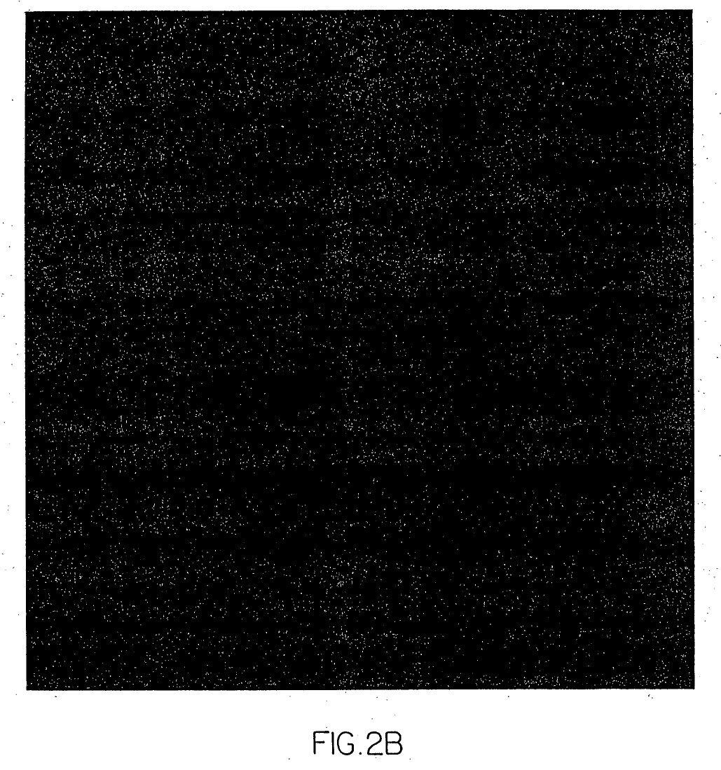

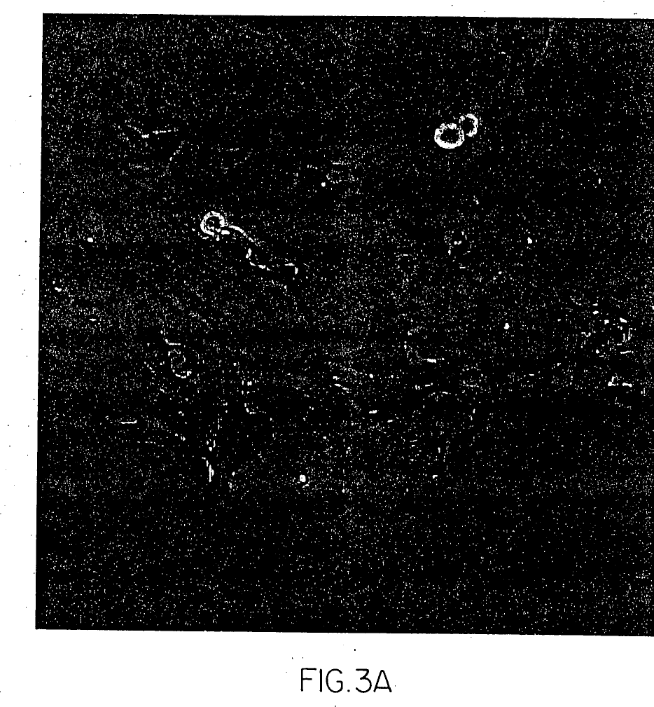

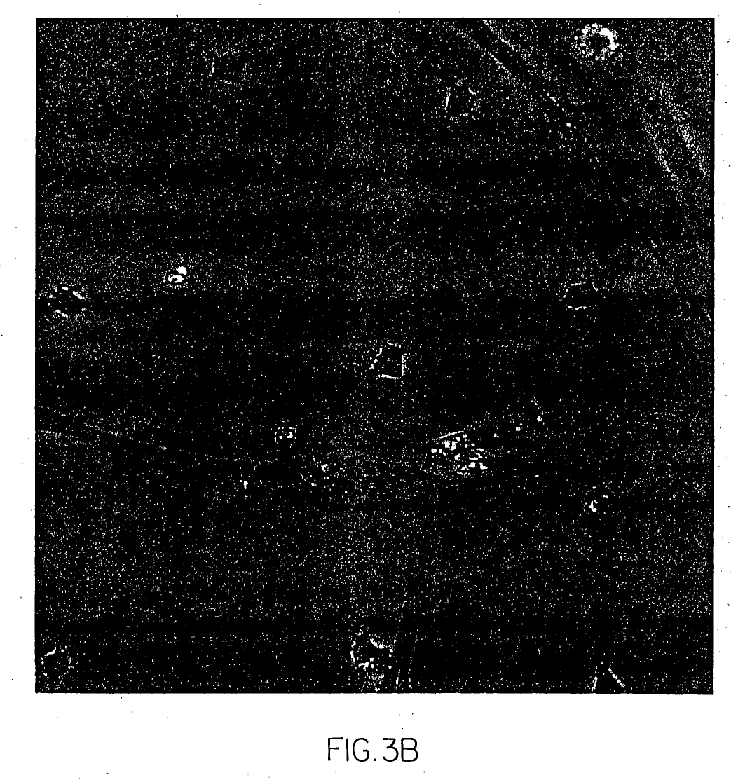

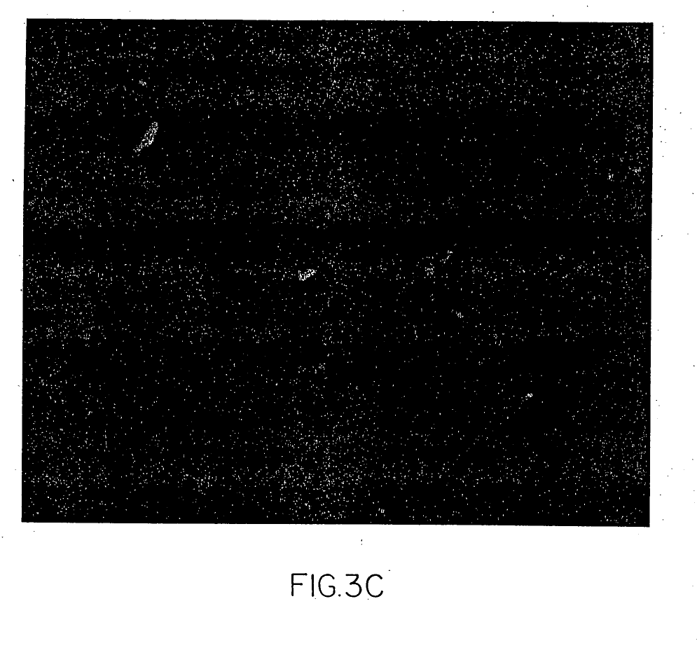

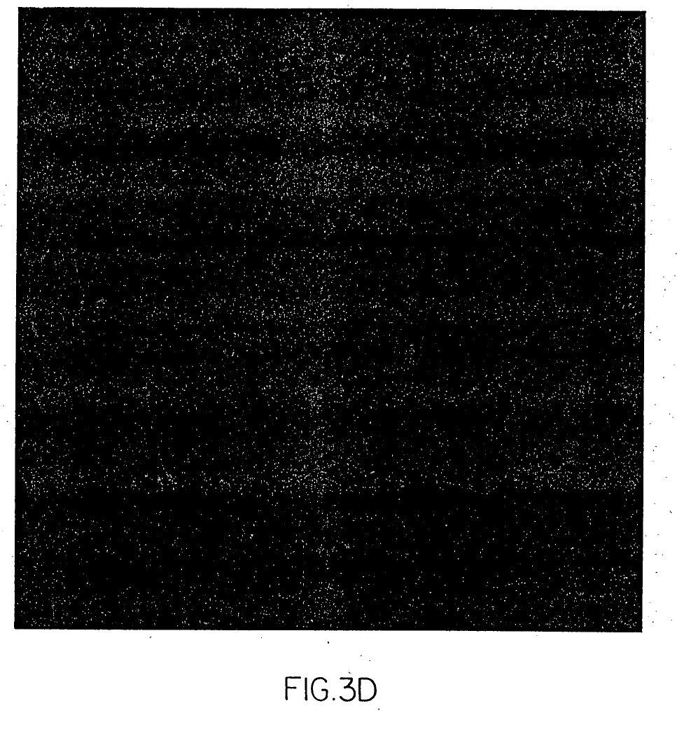

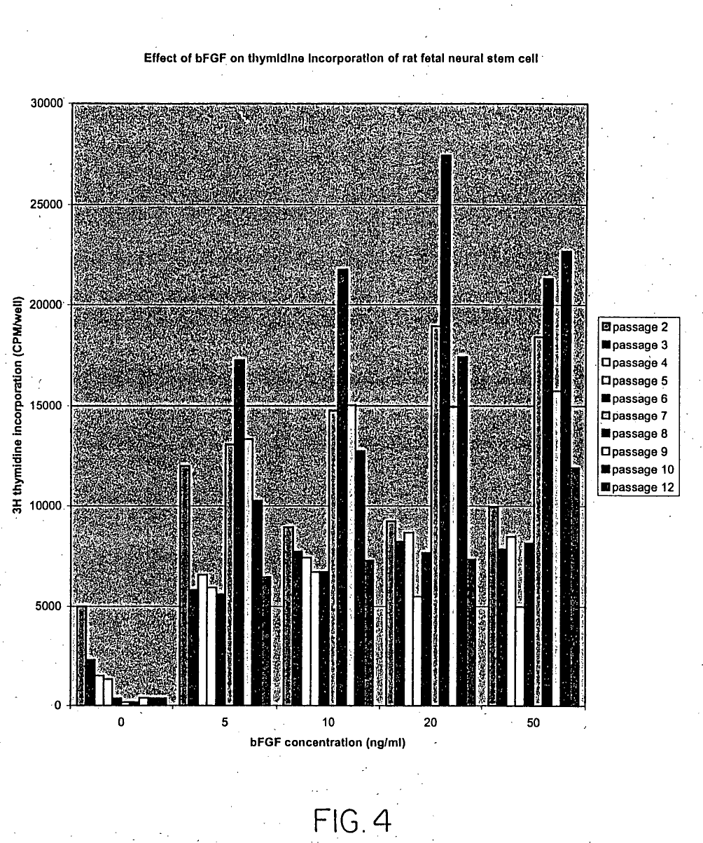

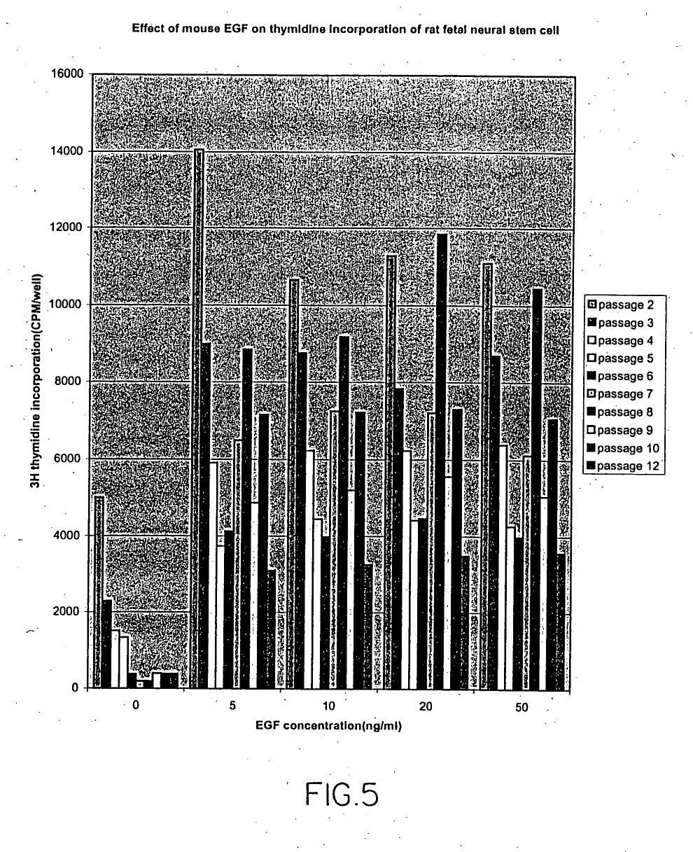

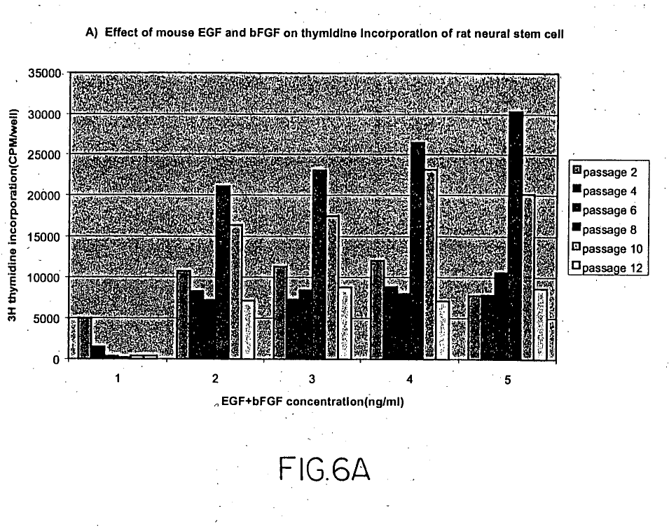

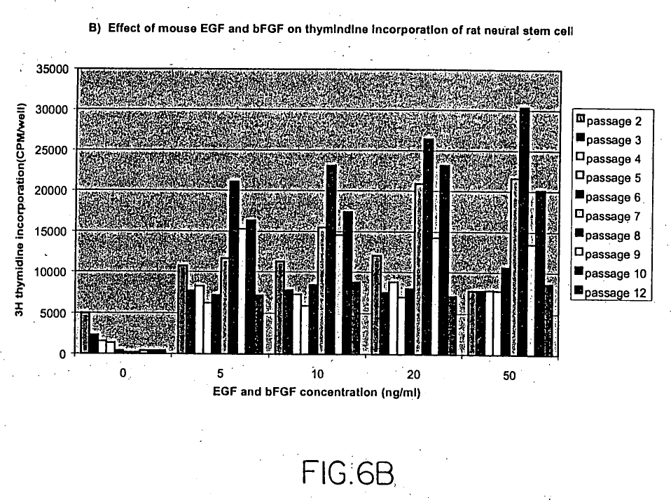

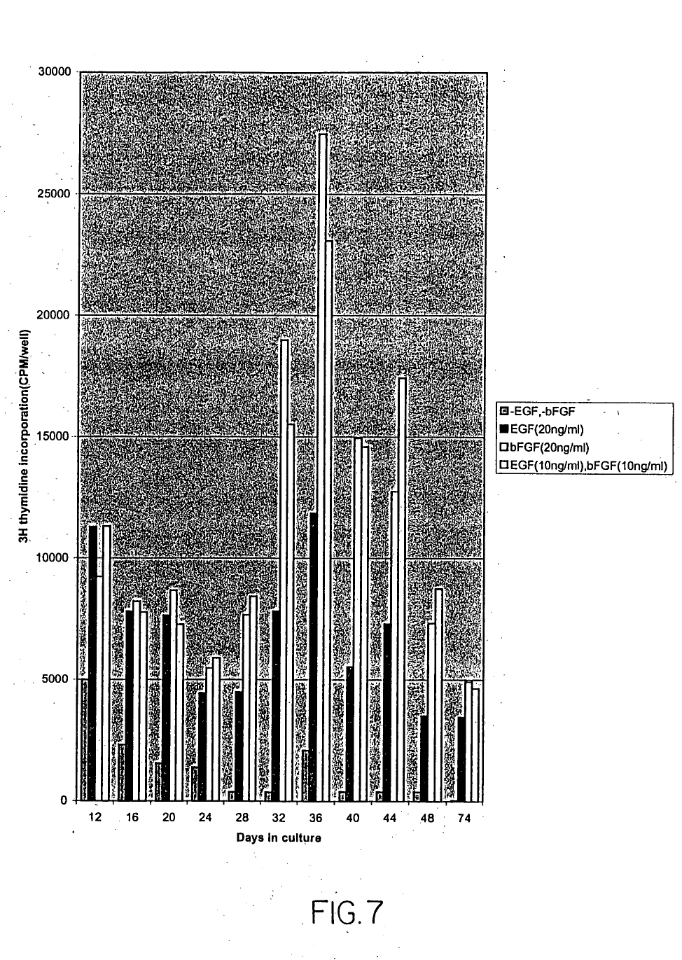

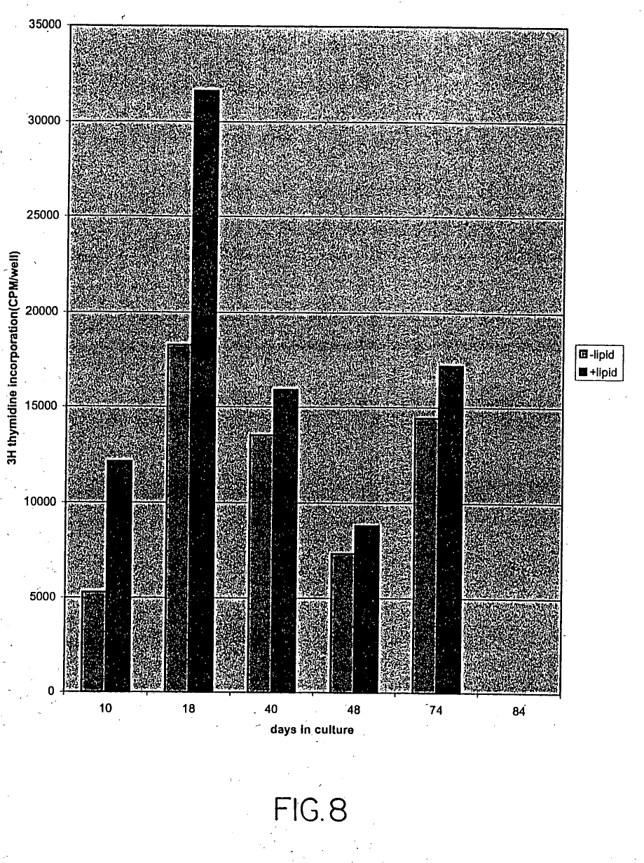

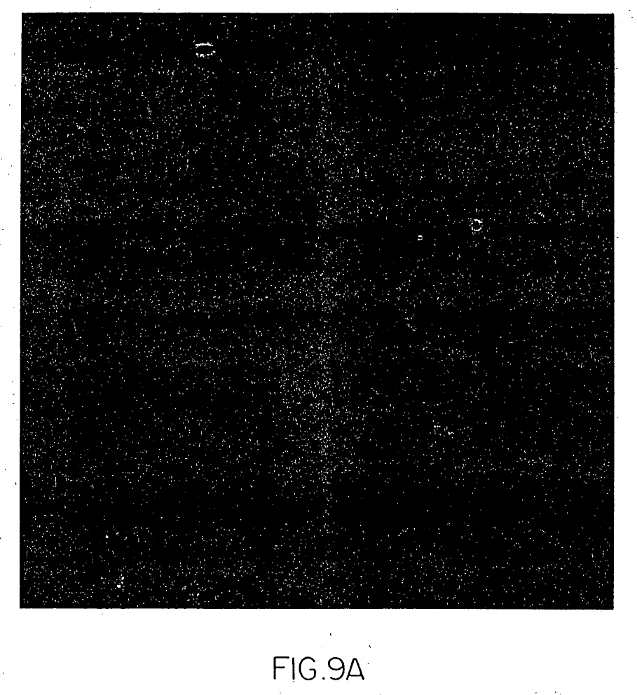

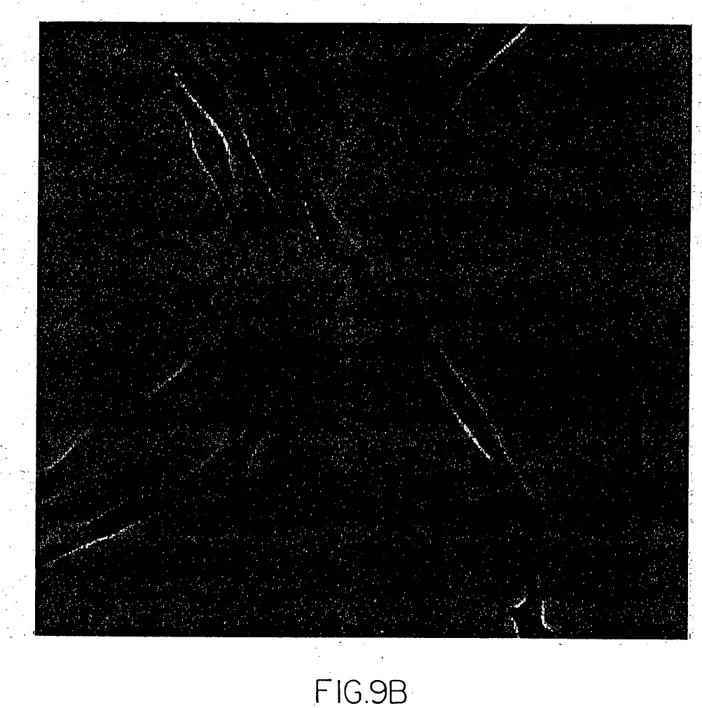

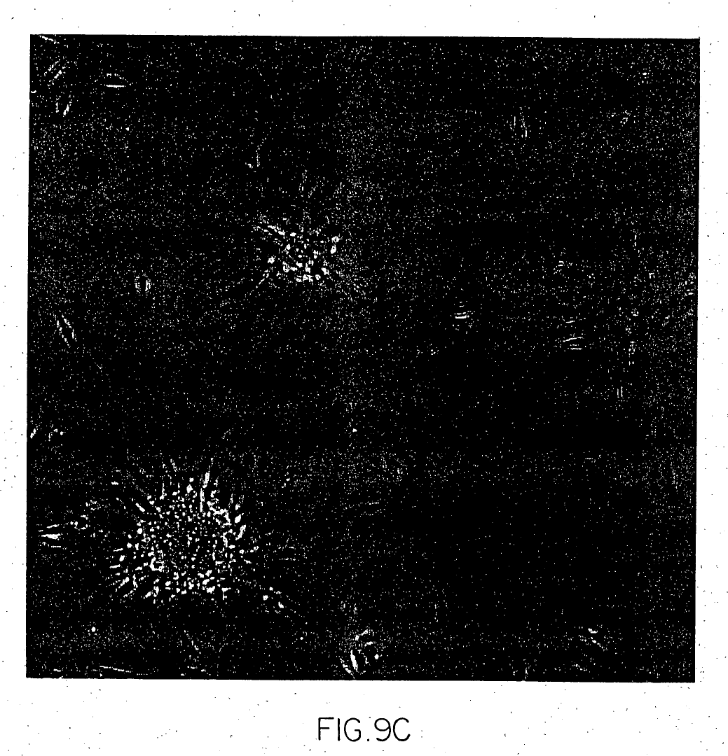

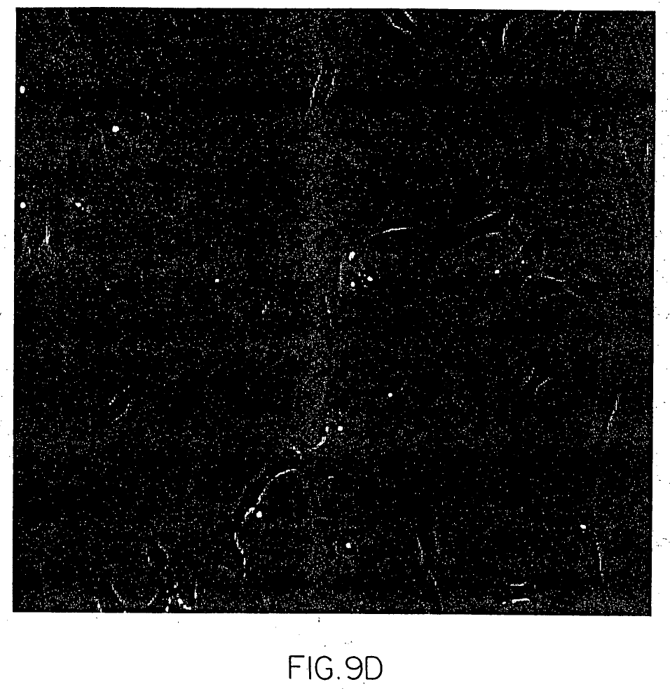

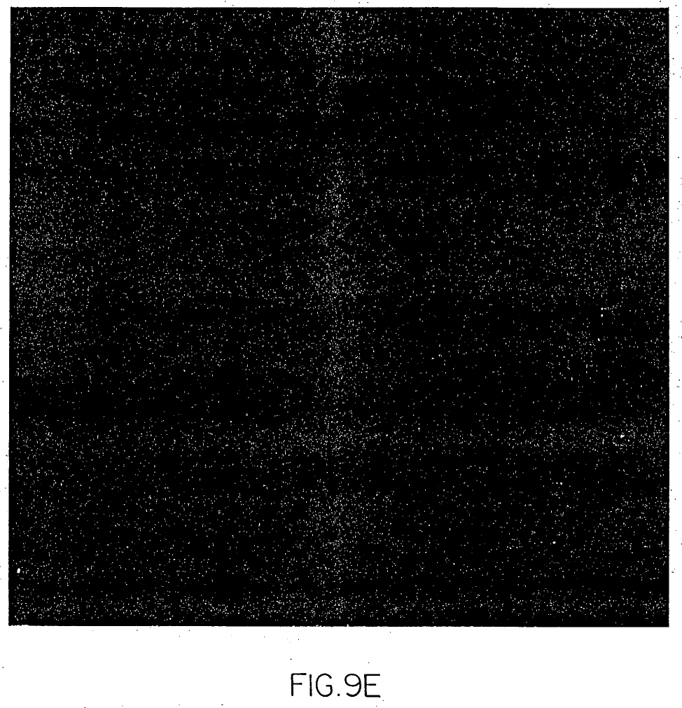

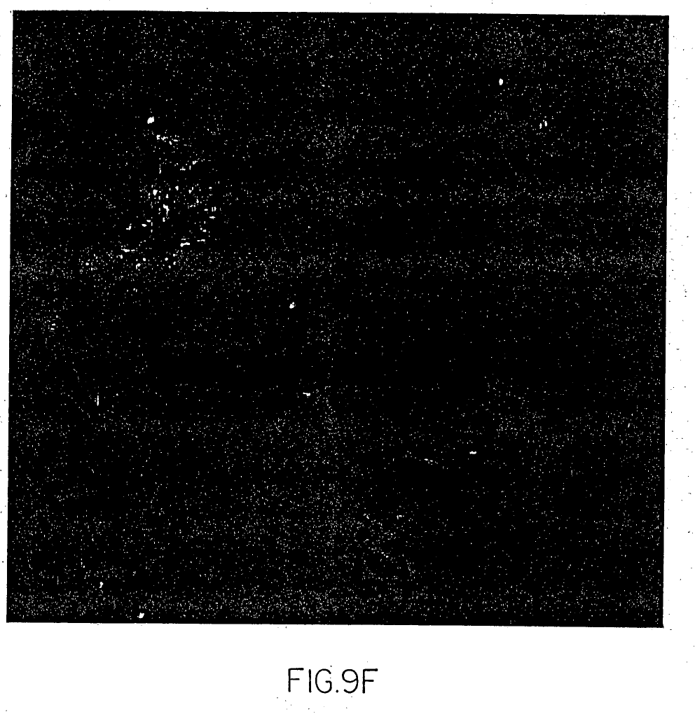

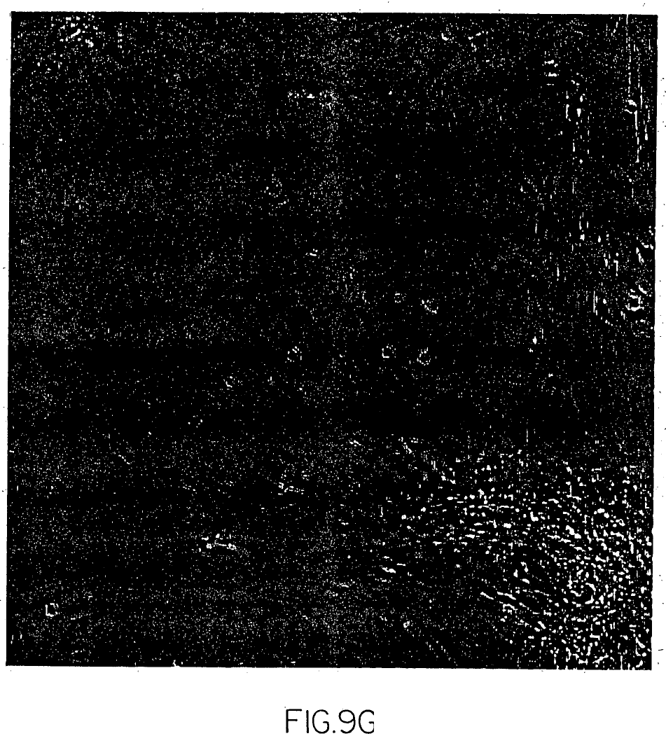

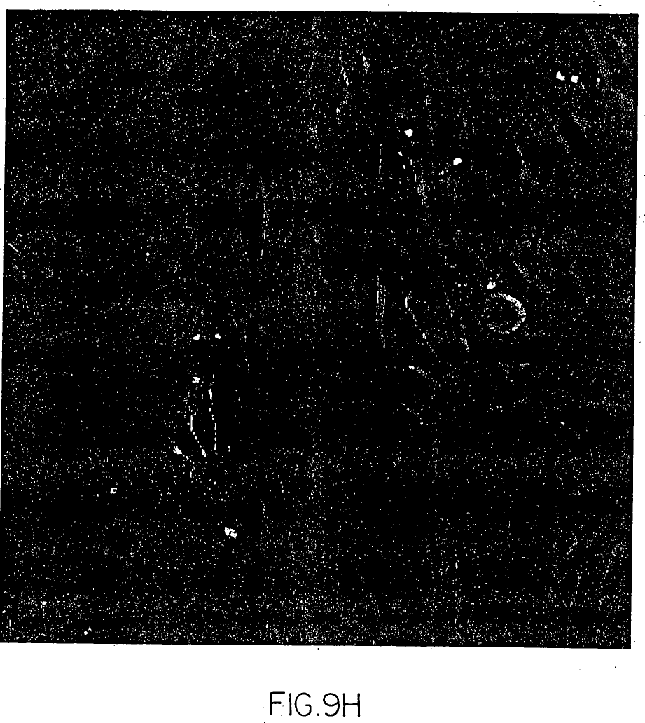

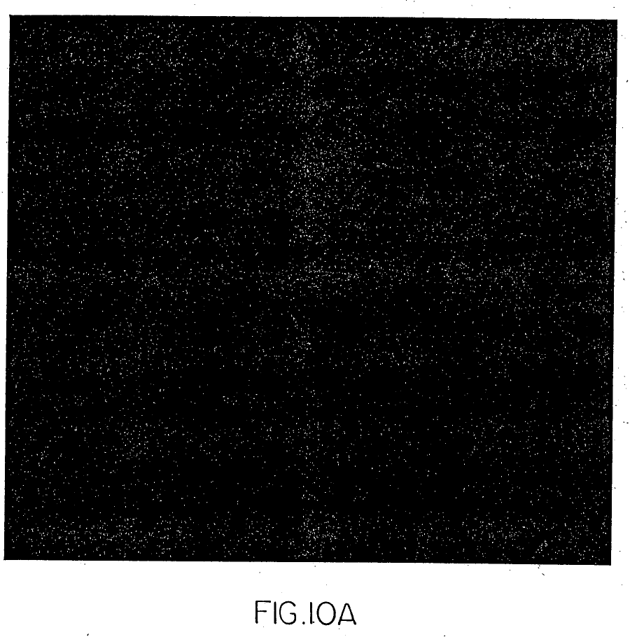

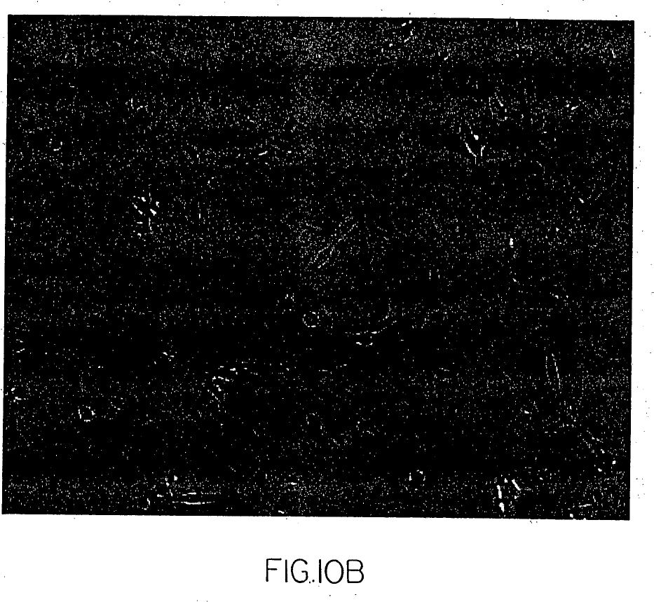

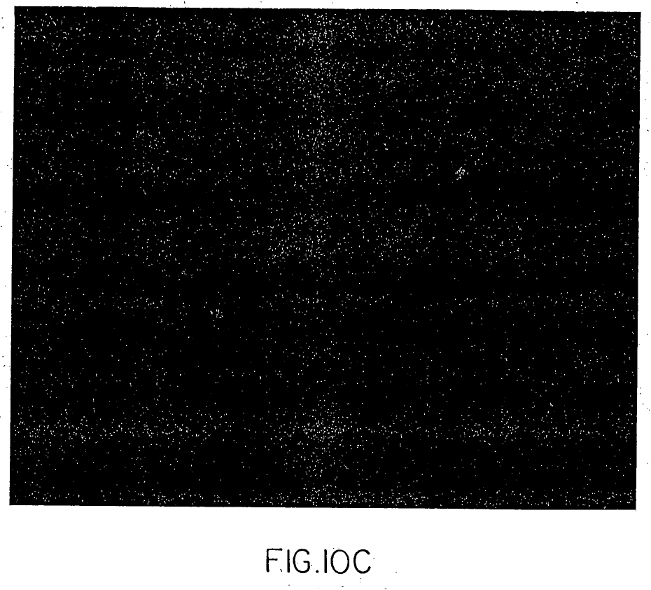

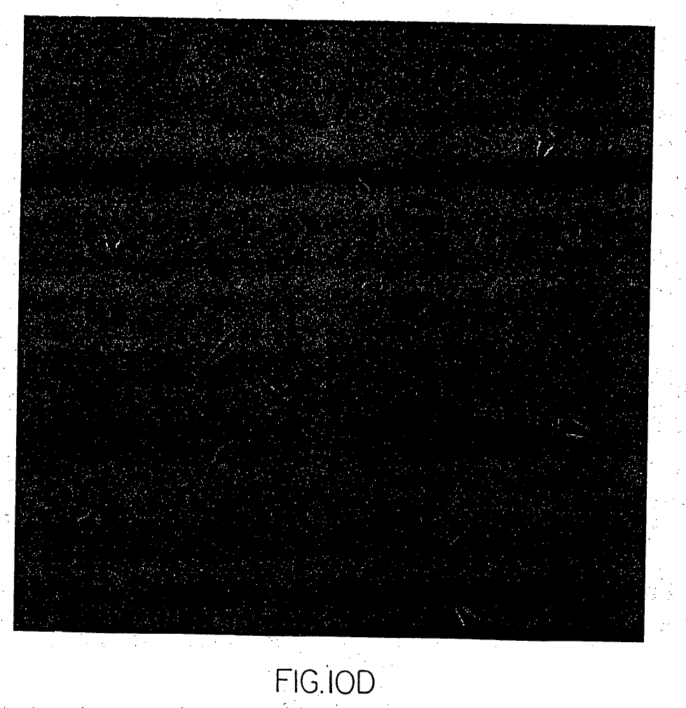

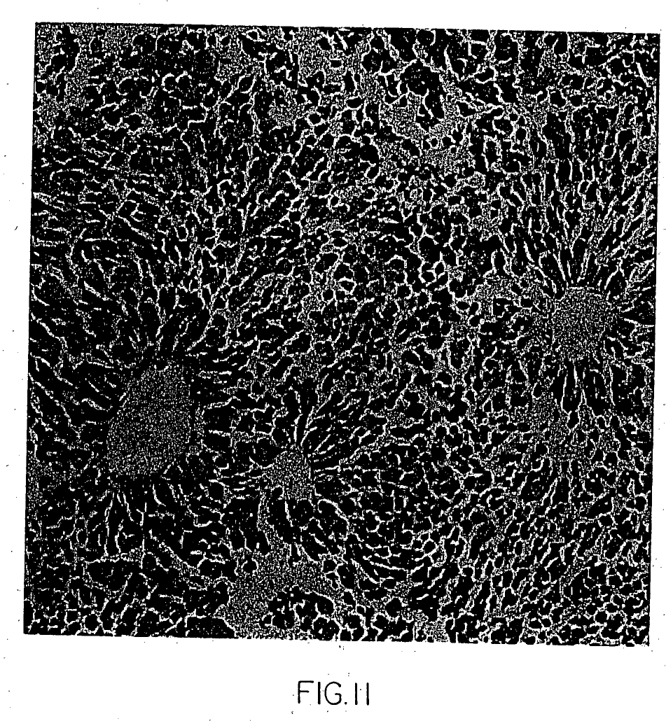

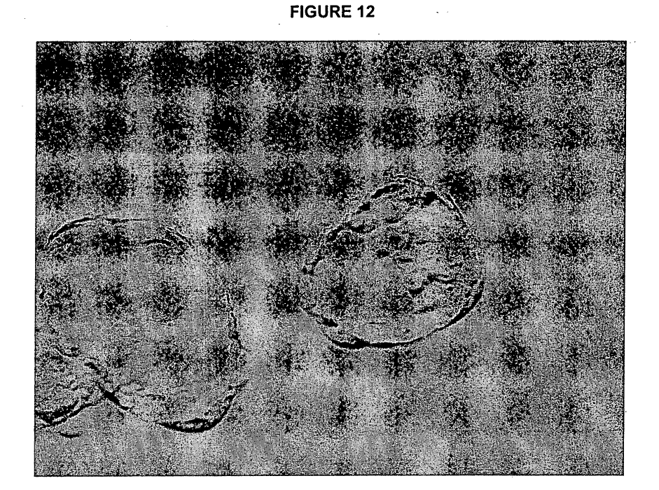

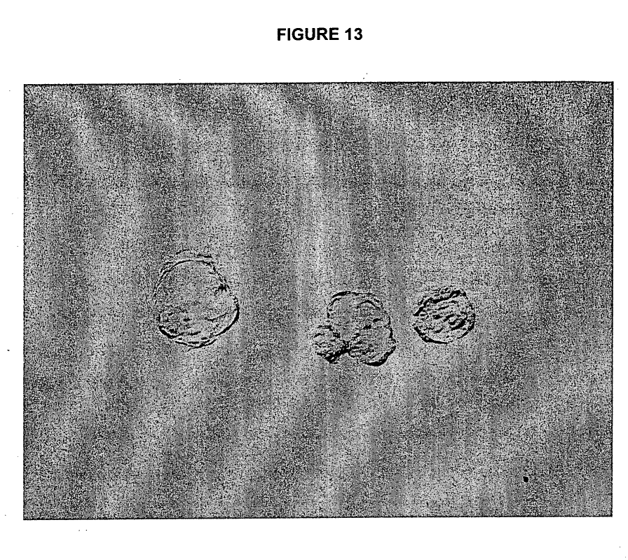

### DESCRIPTION OF THE INVENTION

In a first aspect of the present invention there is provided a cellular composition comprising one or more cells having a property characteristic of a neural stem cell and wherein said neural stem cell is capable of long term culture. Preferably the cells have a property characteristic of a foetal neural stem cell.

The term “long term culture” described herein means an ability to grow indefinitely such that the cell may be passaged to new cultures.

The neural stem cells of the present invention may be characterised by their ability to grow indefinitely in tissue culture without undergoing transformation and retain some degree of developmental plasticity. The phenotype of the neural stem cells do not change over long term culturing and the plasticity of the neural stem cells make them suitable for nuclear transfer experiments and various other applications such as gene knockout experiments.

Like all neural stem cells, or preferably foetal neural stem cells, these cells have the capacity to differentiate into one or more different types of cells when placed in differentiating conditions. The types of cells, which may result from differentiation, include haematopoietic stem cells and their lineages and neural stem cells and their lineages.

The neural stem cells, and preferably the foetal neural stem cells have the capacity to grow indefinitely in tissue culture and this means that they can remain undifferentiated. The degree of plasticity means that these cells have the ability to generate multiple cell types and the cells of the present invention may be identified by these characteristics.

The introduction of telomerase catalytic component (TERT) represents an alternate method for obtaining an immortalised, non-transformed cell line. Accordingly, it is preferred that a somatic cell, more preferably a rat foetal fibroblast are or have been manipulated to express telomerase catalytic component (TERT). However, cells already expressing TERT and which are not genetically modified may be present in the cellular composition. More preferably, the gene encoding TERT is introduced into the cell. This can result in a cell line that is immortalized. The expression of TERT in the cells may also allow the cells to undergo (repeated) genetic manipulations as the cells can be grown continuously in culture for many weeks and/or months. TERT may be inserted into the cell line of choice using standard transfection technologies.

The term “TERT cell(s)” as used herein means a cell which expresses TERT either naturally or by introduction via genetic manipulation. A “TERT cell” is a somatic cell which expresses TERT by introduction via genetic manipulation. More preferably the TERT somatic cell is a TERT foetal fibroblast cell.

The neural stem cells, require the presence of at least one growth factor, preferably epidermal growth factor (EGF) or basic fibroblast growth factor. (bFGF) for cell division. Removal of EGF from the medium stops cell division in the cells and induces quiescence of the cells in the absence of any growth factor such as bFGF or PDGF. Absence of a growth factor does not kill the cells. Depending on the passage number of the cells, the reintroduction of a growth factor may stimulate the cells to re-enter the cell cycle.

Another important feature of the present cells is their capacity to culture indefinitely and “bud off” into the media. This feature can be utilised as a method of propagation of the cells. Each bud comprises a plurality of cells which may be cultured to provide an isolated and purified population of the neural stem cells. Preferably they are foetal neural stem cells.

The cells may also be identified by cell markers. Apart from the standard neural cell markers, other markers including but not limited to nestin, vimentin etc, may be used to identify the neural stem cells, preferably foetal neural stem cells. Accordingly these markers are consistent with the description of the cells as foetal neural stem cells.

Further these cells can be made to differentiate into various neuronal lineages and display markers consistent with differentiated neuronal stem cells, for example, G-FAP, a marker of glial cells, β tubulin, a marker consistent with neurones.

In another aspect of the present invention, there is provided a method of preparing a cellular composition comprising one or more cells having a property characteristic of a neural stem cell and wherein said neural stem cell is capable of long term culture said method comprising:
obtaining a source of neural stem cells;
preparing a suspension of cells from the source;
contacting the suspension of cells with a suitable medium to maintain the neural stem cells in a cell culture; and
culturing the cells including passaging and propagation of cells.

Preferably the neural stem cell is a foetal neural stem cell having the properties as described above.

The source of neural stem cells may derive from any animal that has a nervous system. Preferably the animal is a mammal including but not limited to murine, bovine, ovine, porcine, equine, feline, simian, endangered species, live stock or may derive from marsupials including kangaroos, wombats.

Neural stem cells may be collected from any embryonic stage of development after that the neural stem cells are present. More preferably the source of neural stem cells is from a foetus which is differentiated at a stage after the embryonic stage. The whole foetus or a part thereof containing neural cells may be used as a source of the neural cells. Preferably the head or spinal cord of the foetus provide the source of neural stem cells. More preferably, the head is used as a source of foetal neural stem cells.

Where the neural stem cell expresses TERT to induce immortality, the TERT neural stem cells may also be obtained from an animal which naturally expresses TERT or a genetically modified animal which has been manipulated to express TERT in its somatic cell lineages. TERT cells may be collected from any stage of development of the animal. Preferably the source of TERT cells is from a foetus which is differentiated at a stage after the embryonic stage. The whole foetus or a part thereof may be used as a source of the TERT cells. Preferably the cells are obtained from a rat expressing TERT in its somatic cell lineages.

Preferably the cells are obtained, from rat foetuses and more preferably from the head of a rat foetus. It has been found that foetus obtained from Sprague-Dawley rats provides a reliable source of foetal neural stem cells.

Membranes from foetuses may be removed and their heads separated from their bodies. The pooled foetal heads may be placed into a 100 mm petri dish and the tissue minced with a blunt object such as the tip of a syringe until homogeneous in size. A syringe may be used to aspirate the minced tissue which may be transferred into a tube. The dish can be washed with 5-10 ml PBS and then aspirated into a syringe and pooled into a tube containing tissue.

The minced tissue may be spun down and resuspended in a small volume of media.

The cells may be placed onto fibronectin+poly-_{L}-Ornithine pre-coated plates at a density of approximately 2.5×10⁵ to 5.0×10⁵ cells/cm² and incubated in 5% CO₂ at 37° C.

In another aspect of the present invention, there is provided a media suitable for culturing neural stem cells (NSCs), said media including at least one lipid and at least one mitogenic factor within said media. Preferably the lipid is selected from cholesterol, triglyceride, or phospholipid or a combination thereof. Most preferably the lipid is cholesterol and phospholipid.

A suitable medium to maintain the cells in culture is a medium which can perpetuate the cultured NSCs as herein described, most preferably they are cultured indefinitely.

In yet another aspect there is provided a method of culturing neural stem cells in long term culture, said method comprising culturing the cells in the presence of at least one lipid and at least one mitogenic factor.

The media may contain known components that in combination, support the growth of the cultured neural stem cells or preferably the foetal stem cells. The media may include other nutrients, buffers, hormones, salts, antibiotics, proteins, growth factors and enzymes, Neurobasal-A media® (Life Technologies), containing Insulin-Transferrin-Selenium (Life Technologies)—1:100; EGF 2-20 ng/ml; bFGF 2-10 ug/ml, Chemically defined lipid concentrate (Life Technologies)—1:100; N-2 supplement (Life Technologies) 1:100, B-27 supplement (Life technologies) 1:100, and L-glutamine 1-2 mM.

A medium which contains at least a combination of one or more mitogenic factors and lipids is found to be most preferred for culturing the NSCs, more particularly for culturing the NSCs indefinitely. Suitable mitogenic factors may be selected from the group including, but not limited to, bFGF, EGF and PDGF. These factors may be used alone or in combination with the lipids providing both lipids and mitogenic factors are included in the media. EGF and/or bFGF are mostly preferred as mitogenic factors in the media.

Some components may be substituted for others (eg insulin-like growth factors for insulin; transforming growth factor alpha for epidermal growth factor; bovine serum albumin containing lipids; polylysine for fibronectin; and iron salts for transferrin). Further, other factors might be added to the culture medium, such as tumour promoters, additional hormones and/or growth factors, bovine serum albumin, low concentrations of serum or plasma, or modified plasma preparations with reduced inhibitory activity. Fibronectin might be eliminated from the culture medium formulation to obtain anchorage-independent growth of the present cell lines. Alteration of culture medium components may also allow derivation of sublines of the non-tumorigenic cell lines of the present invention or their equivalent. In addition, other supplements may be added to the medium formulation to enhance protein production from a particular foreign gene construct (for example, addition of steroid hormones where the foreign gene is operably linked to a steroid hormone-responsive promoter).

More preferably, the media contains at least a cell survival factor, such as transferrin, insulin, growth factors such as EGF, bFGF (FGF-2) or PDGF, lipids and selenium.

The foetal neural stem (FNS) cell medium suitable for the present invention preferably comprises Dulbecco-modified Eagle's medium (DMEM) comprising 15 mM 4-(2-hydroxy-ethyl)-1-piperazine-ethanesulfonic acid, 4.5 g/l glucose, 1.2 g/l bicarbonate, 200 U/ml penicillin, and 200 μg/ml streptomycin. The following additional components preferably added prior to use of the media include bovine insulin (10 g/ml), human transferrin (25 μg/ml), mouse EGF (2-20 ng/ml), sodium selenite 10 nM, and human HDL 25 μg/ml. The EGF growth factor may be substituted with bFGF (FGF-2) or any other suitable mitogenic growth factors.

Methods of identifying the cells which have the characteristics of neural stem cells may be any method known to the skilled addressee for detecting the properties listed above. For instance for detecting cell markers, antibodies (monoclonal or polyclonal) are available to identify them.

Methods of isolation may be employed based on the methods of identification. For instance, antibodies may be used to select those neural stem cells having the appropriate markers, alternatively suitable cell culture conditions may be used to obtain cells with the morphology of the neural stem cells of the present invention.

In another aspect of the present invention there is provided a cellular composition comprising a substantially homogeneous population of cells having a property characteristic of a neural stem cell and wherein said cell is capable of long term culture. Preferably the cells have a property characteristic of a foetal neural stem cell.

Preferably, the cellular composition includes somatic cells expressing TERT either naturally or by genetic manipulation.

In another aspect of the present invention, there is provided a method of preparing a cellular composition comprising a substantially homogeneous population of cells having a property characteristic of a neural stem cell and wherein said cell is capable of long term culture said method comprising:
obtaining a source of neural stem cells;
preparing a suspension of cells from the source;
contacting the suspension of cells with a suitable medium to maintain the neural stem cells in a cell culture;
culturing the cells including passaging and propagation of the cells.

The neural stem cells of the present invention have the characteristic of being able to “bud off” into the media. These can be seen with the naked eye. The buds may be collected and spun down. The buds may be disaggregated by any method available to the skilled addressee. However, vigorous pipetting can disaggregated the buds to provide separate cells. Prolonged use of trypsin is discouraged as the cells are sensitive to trypsin. Once disaggregated, the cells may be inoculated into a fresh medium, preferably in a media described above. Therefore the present invention also relates to the long-term clonal expansion or propagation of neural stem cells, preferably foetal neural stem cells.

The cells may be passaged using trypsin for a short period. Cells are first washed with PBS to remove media. The cells may be loosened from the plate using a trypsin solution for a minimal period at 37° C., usually less than 2 min. Preferably the cells be free of the tissue culture plate. However, they do not need to be totally disaggregated. The trypsin may be neutralised using soyabean trypsin inhibitor, preferably at: 1 mg/ml made up in the media being used to culture cells added 1:1 (v/v) to the trypsin solution. The cells may be spun down at low speed in a centrifuge, the media removed and the cells resuspended in fresh media and plated in new fibronectin-treated tissue culture plates. The cells may be split 1:4. Preferably the cells are maintained at a minimum plating density of 2.5×10⁵ to 5.0×10⁵ cells cm². FNS cells have a tendency to differentiate when plated at low density.

The cells may be frozen preferably in Neurobasal A Media containing 7.5% DMSO or by any methods available to the skilled addressee which would be suitable for freezing cells.

The neural stem cells of the present invention have the capacity to grow indefinitely without undergoing transformation and retain a degree of plasticity. This can be achieved by culturing and propagating the cells as described above.

Accordingly, the present invention also provides an isolated neural stem cell prepared by the method described above. Preferably it is a foetal neural stem cell.

In another aspect of the present invention, there is provided a genetically modified neural stem cell, said cell having a destroyed, modified or deleted gene. Such genetically modified neural stem cells are useful in gene targeting and gene knockout experiments.

A genetically modified somatic cell or a genetically modified TERT cell refers to a cell or TERT cell into which a foreign (ie non-naturally occurring) nucleic acid, eg, DNA, has been introduced. The foreign nucleic acid may be introduced by a variety of techniques, including, but not limited to, calcium-phosphate-mediated transfection DEAE-mediated transfection, microinjection, retroviral transformation, electroporation, immunoporation, protoplast fusion and lipofection. The genetically modified cell may express the foreign nucleic acid in either a transient or long-term manner. In general, transient expression occurs when foreign DNA does not stably integrate into the chromosomal DNA of the transfected cell. In contrast, long-term expression of foreign DNA occurs when the foreign DNA has been stably integrated into the chromosomal DNA of the transfected cell.

Foreign (heterologous) nucleic acid may be introduced or transfected into neural stem cells or TERT cells. A multipotent neural stem cell or TERT cell which harbours foreign DNA is said to be a genetically modified cell. The foreign DNA may be introduced using a variety of techniques. In a preferred embodiment, foreign DNA is introduced into multipotent neural stem cells or TERT cells using the technique of retroviral transfection. Recombinant retroviruses harbouring the gene(s) of interest are used to introduce into multipotent neural stem cells or TERT cells using the technique of retroviral transfection. Recombinant retroviruses harbouring the gene(s) of interest are used to introduce marker genes, such as but not limited to βgalactosidase (lacZ) gene, or oncogenes. The recombinant retroviruses are produced in packaging cell lines to produce culture supernatants having a high titre of virus particles (generally 10.sup.5 to 10.sup.6 pfu/ml). The recombinant viral particles are used to infect cultures of the neural stem cells or TERT cells or their progeny by incubating the cell cultures with medium containing the viral particles and 8.μ.g/ml polybrene for three hours. Following retroviral infection, the cells may be rinsed and cultured in standard medium. The infected cells may be then analysed for the uptake- and expression of the foreign DNA. The cells may be subjected to selective conditions which select for cells that have taken up and expressed a selectable marker gene.

The present invention accordingly includes foetal neural stem cells isolated by the methods hereinbefore described which are transfected with exogenous nucleic acid. Selected foreign nucleic acid may be introduced and/or recombinantly expressed in the cells of the present invention through the use of conventional techniques.

In another aspect of the present invention there is provided a method of preparing a genetically modified animal, said method comprising introducing a neural stem cell into an oocyte or embryo and allowing the resulting embryo to mature to a foetus or animal.

The neural stem cell is preferably a foetal neural stem cell prepared by the methods described above. In a preferred aspect the neural stem cell is a genetically modified neural stem cell as described above having a gene inserted, deleted or destroyed. The foreign gene may be a gene encoding a desired product preferably to induce a desired characteristic in the genetically modified animal or to generate a gene knockout model wherein the gene is absent.

Accordingly, the present invention preferably provides knockout animals which are useful for research in gene function, diseases, drug therapies and gene development of animal strains having knockout genes prepared as described above.

In another aspect of the present invention there is provided a method of producing an animal, said method comprising introducing a continuously growing donor cell nucleus from a continuously growing donor cell into an oocyte or embryo and allowing the resulting embryo to mature and to preferably develop to a foetus or animal.

It is desirable to use a donor cell or cells which have the ability to grow continuously in culture. Some cells have the limitation of being short lived and they stop dividing in a very short period. Accordingly there is little time for genetic manipulation of these cells and this is often a major limitation in genetic modification or knockout studies. Some cell lines which are naturally continuously growing (ie neuronal stem cells) and which do not require further genetic manipulation, may also be used. From these cells, the nucleus may also be extracted and used in the present invention. The nucleus may be extracted from neural stem cells described above and preferably grown under conditions utilizing the media as described above.

In a preferred aspect of the present invention, the donor cell is a genetically modified continuously growing somatic cell. Similarly, the nucleus may be derived from a genetically modified somatic cell which is continuously growing. Preferably the nucleus is from a neural stem cell as described above wherein the cell is capable of long term culture and hence is continuously growing. Alternatively, the nucleus is from a foetal fibroblast cell line.

Preferably the donor cell nucleus is derived from a non-transformed cell line. Manipulation or genetic modification of the cell line by any method that immortalizes the cell line may be used. More preferably, the nucleus is from a somatic cell line. More preferably, it is from a foetal fibroblast cell line.

The following description exemplifies a type of cell line which is capable of continuous growth and is suitable as a donor cell in the method for producing an animal. However, it should be appreciated that the invention should not be restricted to this cell line or the nuclei derived from these cells as the invention is applicable to all cell lines capable of continuous growth and immortality. The following description is merely illustrative and should not be taken as a restriction on the generality of the invention.

The expression of telomerase catalytic component (TERT) in a cell may induce the cell to immortalize and undergo continuous growth in culture. Accordingly, it is preferred that the cells are or have been manipulated to express telomerase catalytic component (TERT). However, cells already expressing TERT and which are not genetically modified may be present in the cellular composition. More preferably, the gene encoding TERT is introduced into the cell. This can result in a cell line that is immortalized. The expression of TERT in the cells may also allow the cells to undergo (repeated) genetic manipulations as the cells can be grown continuously in culture for many weeks and/or months. TERT may be inserted into the cell line of choice using standard transfection technologies.

TERT may be cloned from cells expressing this gene (eg embryonic tissue may be used). Alternatively the cDNA for TERT is commercially available.

The TERT cells may also be obtained from an animal which naturally expresses TERT or a genetically modified animal which has been manipulated to express TERT in it's somatic cell lineages. TERT cells may be collected from any stage of development of the animal. Preferably the source of TERT cells is from a foetus which is differentiated at a stage after the embryonic stage. The whole foetus or a part thereof may be used as a source of the TERT cells. Preferably the cells are obtained from a rat expressing TERT in its somatic cell lineages.

Preferably the TERT cell is a TERT somatic cell. The TERT somatic cell may be prepared by the methods described above for long term neural stem cell culture. Such cultures are enhanced by expression of TERT which allows for continuous growth of the neural stem cells. Such cells are particularly useful for nuclear transfer.

Where the TERT cell is a TERT somatic cell, it is preferred to be a TERT foetal fibroblast cell.

Oocytes may be obtained from any source. For example, they may be of bovine, ovine, porcine, murine, caprine, simian, amphibian, equine or of a wild animal origin. Preferably the oocyte is a rodent oocyte. More preferably it is a rat oocyte.

The entire contents of PCT/AU97/00868 are hereby incorporated and referred to in this description particularly with respect to the oocytes suitable for this invention and of the enucleation of suitable oocytes.

The TERT cell or cells or nucleus of the TERT cells may be introduced into the oocyte or embryo using any method available to the skilled addressee. Preferably nuclear transfer procedures are used. More preferably a TERT cell is injected into an enucleated oocyte, the oocyte is activated to initiate development and the resulting embryo is transferred to a receptive recipient animal capable of supporting the development of the embryo into a foetus or animal. Other methods may be used to introduce the cell into an oocyte or embryo including but not limited to aggregation of the TERT cell or cells with preimplantation embryos or injection of the TERT cell or cells into the cavity of a blastocyst stage embryo.

The entire contents of PCT/AU99100275 are hereby incorporated and referred to in this application, particularly for the description of nuclear transfer of donor cells into oocytes.

In a preferred aspect of the present invention, the donor cell is a genetically modified TERT cell, said TERT cell comprising a foreign gene which has been introduced into the TERT cell.

In another preferred aspect, the nucleus is derived from a genetically modified TERT cell comprising a foreign gene which has been introduced into the TERT cell. Preferably the TERT cell is a genetically modified TERT somatic cell. More preferably it is a genetically modified foetal fibroblast cell.

In a preferred aspect of the present invention, the donor cell is a further genetically modified TERT cell, said TERT cell having a destroyed, modified or deleted gene. Such genetically modified TERT cells are useful in gene targeting and gene knockout experiments.

These genetically modified TERT cells include the above genetically modified TERT cell wherein the introduced foreign gene is modified or mutated after genetic modification.

In yet another preferred aspect, the nucleus is derived from a genetically modified TERT cell, said TERT cell having a destroyed, modified or deleted gene.

Any of these genetically modified TERT cells or nucleus derived therefrom may be used in the methods of producing animals described herein.

In another aspect of the invention, there is provided an embryo, wherein said embryo results from introducing a continuously growing donor cell nucleus from a continuously growing donor cell into an oocyte or embryo prepared by the method described herein. The embryo is preferably a transplantation embryo.

The donor cells and the nucleus may be as described above.

In another aspect of the present invention there is provided a method of producing a cell line that may be expanded from an embryo to produce cloned cells of an embryo, said method comprising
introducing a continuously growing donor cell nucleus from a continuously growing donor cell into an oocyte or embryo;
culturing the oocyte or embryo to an advanced cleavage stage embryo;
separating and cloning the cleaved cells of the embryo; and
optionally culturing the cloned cells.

The donor cells and the nucleus may be as described above.

Once the cell lines are cloned, these may be used to generate genetically identical lines and animals. This technique may be particularly useful for non-murine models such as monkeys to develop genetically identical animals.

The cells of such a nuclear transplantation embryo may be recycled to provide donor cells for further cycles of nuclear transfer, as described in australian patent 687422 to the present applicant, the entire disclosure of which is incorporated herein by reference.

Accordingly, in another aspect, the present invention provides a cell line expanded from an embryo as prepared by the methods described herein.

In a further aspect of the present invention there is provided an animal produced by the methods of the present invention. Preferably, the animal is a genetically modified animal, preferably the genetically modified animal is a knockout animal.

The transplantation embryos produced by the methods of the present invention may be used to produce genetically identical or similar animals by transplantation into a recipient female, preferably a synchronised female. Preferably, the recipient female is synchronised using-fertility drugs, steroids or prostaglandins. Methods for transfer of embryos to recipient females are known to those skilled in the art.

A genetically modified animal may include the addition of foreign genes capable of identification by the presence of marker genes which have been introduced into a donor cell or nucleus. Suitable marker genes may include fluorescently labelled genes which may facilitate identification of genetically modified animals. A genetically modified animal may include a transgenic animal.

Genetically modified animals may also include knockout animals having genes targeted, destroyed and/or modified so that an animal is developed without the gene. Genes may be modified by removal from the genome or by point or random mutations in a gene.

Accordingly, the present invention preferably provides knockout animals which may be useful for research in gene function, diseases, drug therapies and gene development of animal strains having knockout genes.

The genetically modified animals may be useful for research purposes at any stage of development, preferably adult knockout animals are obtained. However animals at any stage of development may be used.

Preferably the animal is a mammal including but not limited to murine, bovine, ovine, porcine, equine, feline, simian, endangered species, live stock or may derive from marsupials including kangaroos, wombats. Preferably the animal is a rodent. Most preferably the animal is a rat.

In another aspect of the invention, there is provided a method of treating a neurological disorder, said method comprising introducing a neural stem cell into a host animal to correct the disorder wherein the neural stem cell is capable of replacing neural cells affected by the neurological disorder.

The neural stem cell is preferably a foetal neural stem cell as described above. For treating a neurological disorder where neural cells are destroyed, the neural cells may be capable of regenerating the neural tissue. Alternatively, if a foreign gene encoding a protein beneficial for treating the neurological disorder is inserted into a neural stem cell or preferably a foetal neural stem cell, then the genetically modified neural stem cell may be introduced into the patient in need of regeneration and treatment of the neurological disorder. Preferably, the neurological disorder is Parkinsons disease.

The present invention also includes the use of foetal neural stem cells in a wide range of applications including but not limited to transplantation, nuclear transfer and gene targeting and gene knockout experiments, the generation of transgenic animals and the construction of animal models.

Throughout the description and claims of the specification, the word “comprise” and variations of the word, such as “comprising” and “comprises”, is not intended to exclude other additives, components, integers or steps.

The present invention will now be more fully described with reference to the following examples. It should be understood, however, that the description following is illustrative only and should not be taken in any way as a restriction on the generality of the invention described above.

### EXAMPLES

### Example 1

### Preparation of Foetal Neural Stem Cells

Tissue culture plates were predated with fibronectin at 1 g/ml and poly-_{L}-Ornithine at 15 μg/ml in DMEM/F12 for 2-24 hours at 37° C.; 5% CO₂. (Enough volume was used to cover the surface). The fibronectin/poly-_{L}-Ornithine was aspirated and plates washed with DMEM/F12. This preparation can be stored at room temp for several days.

A pregnant rat (eg. Sprague-Dawley) was humanely killed at 9.5-16.5 days gestation by CO₂ asphyxiation. More preferably the foetuses are obtained at 12.5-14.5 days of gestation. Foetuses were removed and placed into a tube with PBS containing penicillin/streptomycin.

Membranes from the foetuses were removed and their heads were separated from their bodies. The pooled foetal heads were placed into a 100 mm petridish and the tissue was minced with a blunt object (the tip of a syringe) until it was homogeneous in size. A syringe was used to aspirate the minced tissue which was then transferred into a tube. The dish was washed with 5-10 ml PBS and then aspirated into the syringe and pooled into the tube containing the tissue.

The minced tissue was spun down and resuspended in a small volume of media.

The cells were placed onto fibronection+poly-_{L}-Ornithine pre-coated plates at a density of approximately 1.5×10⁵ cells/cm² and incubated in 5% CO₂ at 37° C.

### Example 2

### Preferred Defined Medium for Culturing of Foetal Neural Stem Cells

Neurobasal-A media® (Life Technologies), containing Insulin-Transferrin-Selenium (Life technologies)—1:100; EGF (Life Technologies) 10 ng/ml bFGF (Life Technologies) 10 ng/ml; Chemically defined lipid concentrate (Life Technologies)—1:100; N-2 supplement (Life Technologies) 1:100; B-27 supplement (Life technologies) 1:100, L-glutamine 1 mM; 200 U/ml Penicillin, 200 μg/ml Streptomycin.

### Example 3

### Alternate Defined Medium for Culturing Foetal Neural Stem Cells

The FNS cell medium suitable for the present invention comprises Dulbecco-modified Eagle's medium (DMEM) comprising 15 mM 4(2-hydroxyethyl)-1-piperazine-ethanesulfonic acid, 4.5 g/l glucose, 1.2 g/A Bicarbonate, 200 U/ml Penicillin, 200 μg/ml Streptomycin, and the following additional components are added prior to use of the media:

Bovine insulin (10 μg/ml), Human transferrin (25 μg/ml), Mouse EGF (2-20 ng/ml), Sodium selenite 10 nM, and Human HDL (freshly isolated) 25 μg/ml. The EGF growth factor may be substituted with bFGF (FGF-2) or any other suitable mitogenic growth factors.

### Example 4

### Preferred Method for Culturing and Passaging of Foetal, Neural Stem Cells

When the cells were cultured onto fibronection+poly-_{L}-Ornithine pre-coated plates a complete change of media was performed daily until cells reached approximately 80% confluency. The media was then aspirated and a small volume of Hanks Buffered Saline Solution (HBSS—Life Technologies) was added to the flask. Cells were harvested with a cell-scraper and transferred to a tube for centrifugation at 800 g for 5 minutes. The cell pellet was resuspended in a small volume of Neurobasal A media and live cell number estimated using a haemocytomer and staining of the cells with Trypan Blue. The cells were placed onto fibronection+poly-_{L}-Ornithine pre-coated plates at a density of approximately 2.5×10⁵ to 5.0×10⁵ cells/cM² with a suitable volume of preferred defined culture medium.

### Example 6

### Alternate Method for Culturing and Passaging of Foetal, Neural Stem Cells

When the cells were cultured in the absence of fibronection+poly-_{L}-Ornithine they adhered loosely, forming colonies of neuronal cells that “bud off” into the media. These neurospheres can be seen with the naked eye. A half media change was carried out every 2-3 days until the attached cells had attained ˜80% confluency. Until then the media, (containing the spheres), was pipetted off and centrifuged at 800×g for 5 minutes. This media was retained for diluting 1:1 with fresh media. The spheres were disaggregated in a small volume of media by pipetting vigorously (with care not to cause bubbles). The disaggregated cells were then inoculated into fresh flasks at a dilution of approximately 1 in 3. Once the adherent cells had reached 580% confluency, the media containing the spheres was pipetted off into a tube. The adherent cells were harvested in HBSS with a cell-scraper and transferred to the same tube. The cells were centrifuged at 800×g for 5 minutes and resuspended in a small volume of Neurobasal A medium. After disaggregation, live cell number was estimated with a haemocytomer and staining of the cells with Trypan Blue. The cells were then plated into fresh flasks at a density of 2.5×10⁵-5.0×10⁵ cells/cm² with a suitable volume of preferred defined culture medium.

### Example 6

### For the Long Term Storage of the FNS Cells

The cells were frozen down in defined Neurobasal A media containing 7.5% DMSO.

### Example 7

### Examination of the FNS Cell Lines for Tumorigenic Capacity

2 SCID mice were inoculated with 5×10⁵ PC12 (rat phaeochromocytoma cells), 2 SCID mice were inoculated subcutaneously with 5×10⁵ rat neural stem cells (passage #12, representing 3 months of continuous culture). Animals were observed weekly. Nineteen days later, mice inoculated with PC12 cells were humanely killed; these had large lesions at all injection sites. Tumours were examined histologically. At 13 weeks mice inoculated with rat FNS cells show no lesion at injection site and remain healthy.

### Example 8

### Assessment of FNS Cell Proliferation Using BrdU Incorporation

NSCs were plated down at a density of A) 2×10⁴/Cm² for passage #2 FNS cells and B) 1×10⁴/cm² for passage #17 cells (representing 4 months of continuous culture). After 3 days of growth in the Neurobasal A media (with recommended supplements) the cells were pulsed with BrdU for 2 hr. They were then fixed with Bouins for 15 min, rinsed with 70% ETOH four times, then treated with 6N HCl in PBS with 1% Triton X at 23° C. for 15 mins. This solution was then neutralised with 0.5M Na Borate in PBS with 1% Triton X for 10 mins at RT. Non specific binding was blocked for 1 hr with 50% goat serum, then mouse monoclonal ant BrdU (Sigma) was put on the cells at 1:400 for 1 hr at 23° C. in 10% goat serum. The second antibody was FITC conjugated goat anti mouse (Sigma) at 1:500 overnight at 4 degrees. Cells were coverslipped with fluorescent mounting medium.

### Example 9

### Media for Growing Rat Foetal Fibroblasts

F12 nutrient media (Life Technologies) containing 10,000 U of penicillin and 500U streptomycin, 15% foetal calf serum (ES cell grade, Life Technologies) was used for the culture and propagation of foetal fibroblasts. This basis media is designated F12/FCS media.

### Example 10

### Preparation of Fibroblasts Cells

A pregnant rat (eg. Sprague-Dawley) was humanely killed at 10.5-16.5 days gestation by CO₂ asphyxiation. Foetuses were removed and placed into a tube with PBS containing penicillin/streptomycin.

Membranes from the foetuses were removed and their heads were separated from their bodies. The pooled carcasses were placed into a small dish (6 cm) and the tissue was minced with a blunt object (the tip of a syringe) until it was homogeneous in size. A syringe was used to aspirate the minced tissue which was then transferred into a tube. The dish was washed with 5-10 ml PBS and then aspirated into the syringe and pooled into the tube containing the tissue.

The minced tissue was left to settle at the bottom of the tube for a few minutes and was carefully aspirated off the liquid. The tissue was washed with fresh PBS until it was reasonably clear (approximately 2 washes). 5 ml of trypsin 0.1% in versene, was added to the tissue and the tube was placed into a 37° C. water bath, for no longer than 15 min (The tubes were mixed occasionally). The tissue was allowed to settle down to the bottom of the tube and the cell suspension was transferred into a centrifuge tube. The tissue was washed in 5 ml F12 media containing FCS, and the cell suspension was pooled with the trypsin cell suspension. Cells are then plated on a standard tissue culture flask and allowed to proliferate. Cells are propagated in F12 media containing FCS according to standard procedures.

### Example 11

### Preparation of TERT Fibroblasts

A mammalian expression vector expressing TERT may be obtained using standard cloning procedures, familiar to anyone experienced in the art, Alternately the TERT expression vector is commercially available.

For stable transfection experiments vectors are linearised at unique restriction endonuclease site. Transfection experiments were initiated on day 3 of culture in 10 cm dishes using Lipofectamine® Plus. Transfection involved addition of 0.1-20 μg of linearised plasmid to 20 μl of Plus® reagent in 750 μl of serum-free (SF) media with incubation at 23° C. for 15 minutes. 30 μl of Lipofectamine® was then added to 720 μl of F12/FCS media and the solutions were then mixed together and incubated at 23° C. for a further 15 minutes. Media was then aspirated from the cells and replaced with 5 ml of SF media. The DNA/Lipofectamine® solution was then added to the cells followed by the addition of 6.5 ml of F12/FCS 2-3 hours later. On the following day media was replaced with F12/FCS media containing a selectable marker (that was included in the original TERT construct) For example in our experience 300 μg/ml of Geneticin® (Gibco BRL Life Technologies) or 50 μg/ml of hygromycin are suitable concentrations for the rat foetal fibroblasts Antibiotic selection was continued for a period of 10 days (ie. Day 14). Following this initial selection processes the cells are maintained on 0.5× the original concentration of antibiotic.

### Example 12

### Nuclear Transfer Using Fibroblast Cells as Donor Nuclei

Animals were killed by decapitation and the oviducts removed in less than 5 minutes. Oviducts were collected into prewarmed calcium free phosphate buffered saline (PBS). Oocytes were liberated from the oviducts into M16 culture medium containing 40 IU/ml hyaluronidase at 37° C. using fine forceps. Oocytes were washed twice in M2 medium after 5 minutes exposure to hyaluronidase. Cumulus free oocytes were transferred to equilibrated modified rat embryo culture medium (MR1ECM) and incubated in humidified 5% CO₂ in air at 37° C. until use.

Oocytes at the metaphase II stage (i.e. with the first polar body extruded) were selected for nuclear transfer (NT).

Oocytes were enucleated in handling media containing cytochalasin B (7.5 μg/ml, Sigma) by gentle aspiration of the polar body and metaphase plate in a small amount of cytoplasm using a glass pipette (inner diameter: 10-15 μm).

After mechanical disruption of the donor cell membranes in Hepes buffered TCM199 with 5% rat serum (199HF) using the injection pipette, the fibroblast nuclei were injected directly into the oocyte cytoplasts. The reconstructed embryos were transferred back into MR1 ECM until activation.

Artificial activation was induced 4 hours after injection by exposing the oocytes to 8% ethanol in phosphate buffered saline for 5 minutes, prior to culture in MR1ECM containing 35CM cychloheximide for five hours.

Embryos were cultured in modified MR1ECM culture media (Oh et al, (1998) Biol Reprod. 59: 884-889) supplemented with 10% Rat Serum in a 5% CO₂ incubator at 37° C.

Embryos were transferred back to primed recipient animals on day 2, 3 or day 4 of culture.

The above example is also applicable for the TERT fibroblasts prepared as in Example 11.

### Example 13

### Results from Nuclear Transfer Experiments Using Transfected Fibroblasts and FNS Cells

Methods for nuclear transfer of fibroblast or FNS cell nuclei are as detailed in Example 12

Donor Cell Type

| | Transfected | |
|---|---|---|
| | Embryonic | Neural stem |
| | Fibroblast | cells |
| | (%) | (%) |
| Oocytes | | |
| oocytes undergoing | 1256 | 317 |
| nuclear transfer | | |
| Survived transfer | 106 (8.4)^{b} | 80 (30.5)^{b} |
| Cleaved to 2-cell | 24 (22.6)^{b} | N/A |
| embryo | | |
| Embryos | | |
| Transferred to Mice | 7 | nil |
| Transferred to Rats | nil | 78 |
| Developing to | 1 (14.3)^{a} | nd |
| Morula/Blastocyst | | |
| Producing Live Born | nd | 0 |

Significant differences in reconstructed embryo survival, cleavage and development in vivo between donor cell types are indicated by different superscript letters (a-b). Relative percentages surviving each manipulation are shown in parentheses. nd: not determined.

Finally, it is to be understood that various other modifications and/or alterations may be made without departing from the spirit of the present invention as outlined herein.

### Example 14

### Evidence of NT Embryo Development in the Rat

The NT embryos were prepared as described in Example 12 except that, in some cases where indicated, NEP (neural epithelial cells) were used. NEP cells are neural epithelial cells which are a neural stem cell (nestin Positive) but are collected at a slightly earlier stage than the FNS cells as herein described. Oocytes were collected from donor animals of varying age as indicated in Table 2. This varied between 4 and 8 weeks.

(a) Preparation of rat NEP Cells from day E10.5 Neural Tubes

Two (2) E10.5 females were killed with CO₂. The uterus was removed and placed in a 10 cm Petri dish containing 20 mL of ice-cold PBS. Under a dissecting microscope, the uterine wall was cut and the embryos removed. The head, the tail and the heart were removed along with most non-neural tissue.

Trunk segments were collected and transferred to a concave glass dish containing ice-cold PBS. The PBS was removed and 1 mL of enzymatic solution (5 mL HBSS (+Ca⁺², +Mg⁺²), 10 mg dispase II, 5 mg collagenase, 1000 U Dnase (0.001%) was added. The tissue was incubated at room temperature for 5-10 mins and the trunks triturated with a Pasteur pipette.

The enzymatic solution was replaced 1-2 times to facilitate digestion. As soon as the ends of the tubes appeared to separate from connective tissue, the enzymatic solution was replaced with 1 mL NEP basal medium (250 mL DMEM/F12 medium, 2.5 mL N2 supplement (100× stock), 5 mL B27 supplement (50× stock; without vit. A), 400 ng bFGF (final concentration: 20 ng/mL), 2.5 mL pen/strep (100×solution), 250 mg BSA (final concentration: 1 mg/mL)). The tubes were washed several times and individual tubes collected (in a minimal volume) and transferred with a Pasteur pipette into a 1.5 mL Eppendorf tube.

Tissue was digested with trypsin and neutralised. The cells were collected by centrifugation. The cells were then resuspended in 100 μL NEP basal medium containing 250 mL DMEM/F12 medium, 2.5 mL N2 supplement (100× stock), 5 mL B27 supplement (50× stock; without vit. A), 400 ng bFGF (final concentration: 20 ng/mL), 2.5 mL pen/strep (100×solution), 250 mg BSA (final concentration: 1 mg/mL). Just before using the NEP basal medium, 18 ml of NEP was added which included+2 mL (Chicken embryo extract) CEE (final concentration: 10%), 400 ng bFGF (final concentration: 20 ng/mL).

The cells were dissociated by very gentle trituration 1-2 times and plated at high density on fibronectin-coated dish (for 2 litters-6 cm or T25 flask; for 1 litter-3 cm PD) in NEP basal medium+1% FCS and incubated at 37° C. in a 5% CO₂ If convenient, the medium was changed to NEP basal medium serum-free after 1-2 hours. If not, the medium was changed next morning.

(b) Preparation of Chicken Embryo Extract (CCE)

CCE was prepared from 11 day old fertilized eggs. The eggs were chilled to render the embryos unconscious. The embryos were removed under sterile conditions and macerated in DMEM. Hyaluronidase was added to a final concentration of 0.02% and incubated at 4° C. for 45 min. The cells were removed and the supernatant (CCE) collected by centrifugation.

(c) Identification of the NEP

NEP cells were identified by their immunocytochemistry: They have the following characteristics:

| | |
|---|---|
| Nestin: | +ve |
| E-NCAM: | −ve |
| A2B5: | −ve |
| β-III tubulin: | −ve |

Nuclei of the NEP may have, in some cases where indicated, been remodelled by chromatin transfer (CT) prior to cloning by nuclear transfer. The technology is described in Sullivan E J et al (2004) Cloned Calves from Chromatin Remodelled In Vitro, Biology of Reproduction, 70, 146-153. The system involves permeabilization of the donor cell and chromatin condensation in a mitotic cell extract to promote removal of nuclear factors solubilized during chromosome condensation. The condensed chromosomes were transferred into enucleated oocytes prior to activation. The oocytes were activated using cytochalasin B (CB) or cytochalasin D (CD).

The embryos were transferred on the day of manipulation into recipient female rats and allowed to develop to embryonic day 12.5.

Table 2 (below) provides a detailed description of experiments performed and their outcomes. Evidence of fetal development was confirmed due to the presence of implantation scars.

Further direct evidence was obtained by collection of embryos at embryonic day 4 and the in vitro culture of the embryos allowing them to develop into blastocysts. The blastocysts developed further in culture as evidenced by embryo hatching (FIGS. 12 & 13). Just prior to implantation, the developing embryo must “hatch” out of its outer shell (zona pellucida). Some embryos seem to have a thicker shell that may decrease their ability to implant. Clearly, these embryos are available for implantation.

TABLE 2
Summary of Nuclear Transfer Experiments

| Donor | Activation | | | No. egg | |
|---|---|---|---|---|---|
| age | reagent | Cell | CT | transferred | Implantation |
| 8 wks | Sr²⁺(1.25 mM) | NEP | No | 82 | 6(7%) |
| | CB | | | | |
| | CHX | | | | |
| 4 wks | Sr²⁺(1.25 mM) | NEP | No | 160 | 4(3%) |
| | CB | | | | |
| | CHX | | | | |
| 4 wks | Sr²⁺(1.25 mM) | Fibroblast | No | 196 | 6(3%) |
| | CB | | | | |
| | CHX | | | | |
| 4 wks | Sr²⁺(1.25 mM) | NEP | No | 70 | 8(11%) |
| | CB | | | | |
| | CHX | | | | |
| 4 wks | Sr²⁺(0.5 mM) | Fibroblast | No | 43 | 5(12%) |
| | CB | | | | |
| | CHX | | | | |
| 4 wks | Sr²⁺(0.5 mM) | NEP | Yes | 183 | 6(3%) |
| | CB | | | | |
| | CHX | | | | |
| 4 wks | Sr²⁺(0.25 mM) | NEP | Yes | 233 | 4(2%) |
| | CB | | | | |
| | CHX | | | | |
| 8 wks | A23187(1.25 μM) | NEP | Yes | 97 | 3(3%) |
| | CHX | | | | |
| | CD | | | | |
| 4 wks | A23187(1.25 μM) | NEP | Yes | 140 | 4(3%) |
| | CHX | | | | |
| | CD | | | | |
| 4 wks | A23187(2.0 μM) | NEP | Yes | 94 | 3(3%) |
| | CHX | | | | |
| | CD | | | | |
| 4 wks | A23187(2.0 μM) | NEP | Yes | 44 | 5 Blastocysts |
| | CHX | | | (in vivo | (Collected |
| | CD | | | culture) | on day 4.) |

1-44. (canceled)

45. A method of producing a non-human embryo, said method comprising introducing a nucleus from a neural stem cell (NSC) into an oocyte and allowing the oocyte to mature to the non-human embryo.

46. The method according to claim 45 wherein the NSC is a fetal NSC (FNSC).

47. The method according to claim 45 wherein the NSC is a telomerase catalytic component (TERT) NSC.

48. The method according to claim 45 wherein the NSC is a telomerase catalytic component (TERT) FNSC.

49. The method according to claim 45 wherein the NSC is capable of long term culture and is derived from a cellular composition prepared by a method comprising:
obtaining a source of neural stem cells;
preparing a suspension of cells from the source;
contacting the suspension of cells with a suitable medium to maintain the neural stem cells in a long term cell culture; and
culturing the cells in the long term culture, wherein said culturing comprises passaging and propagation of the cells.

50. The method according to claim 49 wherein the long term culture is a period of 4 to 6 weeks.

51. The method according to claim 49 wherein the source of the neural stem cell is a fetus differentiated at a stage after the embryonic stage.

52. The method according to claim 51 wherein the source of the neural stem cell is a head or spinal cord of the fetus.

53. The method according to claim 49 wherein the suitable medium includes at least one lipid and at least one mitogenic factor.

54. The method according to claim 53 wherein the lipid is selected from the group consisting of cholesterol, triglycerides or phospholipids or a combination thereof.

55. The method according to claim 53 wherein the mitogenic factor is selected from the group consisting of bFGF, EGF, PDGF or a combination of EGF and bFGF.

56. The method according to claim 55 wherein the EGF is in the range of 2 to 20 ng/ml.

57. The method according to claim 55 wherein the bFGF is in the range of 2 to 20/ml.

58. The method according to claim 53 wherein a chemically defined lipid concentrate is present in a ratio of 1:100.

59. The method according to claim 53 wherein the media further includes a cell survival factor.

60. The method according to claim 59 wherein the cell survival factor is selected from the group consisting of transferrin, insulin, growth factors including EGF, bFGF (FGF-2) or PDGF, lipids and selenium.

61. The method according to claim 49 wherein the passaging and propagation of the cells is conducted when the cells bud from the cell culture.

62. The method according to claim 45 wherein the NSC is genetically modified and wherein the genetic modification comprises destroying, modifying or deleting a gene.

63. A method of producing a genetically modified non-human animal said method comprising:
obtaining an embryo prepared by the method according to claim 45; and allowing the embryo to mature to the genetically modified non-human animal.

64. A method of producing a genetically modified non-human animal said method comprising:
obtaining an embryo prepared by the method according to claim 62; and allowing the embryo to mature to the genetically modified non-human animal.

65. A method of producing a cell line from an embryo to produce cloned cells of an embryo, said method comprising:
obtaining an embryo prepared by the method according to claim 45; culturing the embryo to an advanced cleavage stage embryo; and
separating and culturing the cleaved cells of the embryo.

66. A method of producing a cell line from an embryo to produce cloned cells of an embryo, said method comprising:
obtaining an embryo prepared by the method according to claim 62; culturing the embryo to an advanced cleavage stage embryo;
separating and culturing the cleaved cells of the embryo.
